(19) United States
(12) Patent Application Publication
(10) Pub. No.: US 2009/0182350 A1
(43) Pub. Date: Jul. 16, 2009

(54) Instrucment and method for treatment of hemorrhoids

(76) Inventor: George Percy McGown, Brooklyn, NY (US)

Correspondence Address: COLEMAN SUDOL SAPONE, P.C., 714 COLORADO AVENUE, BRIDGE PORT, CT, 06605-1601, US

(21) Appl. No.: 12/009,083

(22) Filed: Jan. 16, 2008

(51) Int. Cl. A61B 17/128; A61B 17/28

(52) U.S. Cl. 606/140; 606/205

(57) ABSTRACT

A medical grasper for treatment of hemorrhoids has an elongate rigid tubular member having a proximal end and a distal end and a bend located between the proximal end and the distal end. An elongate shifter element extends longitudinally into the tubular member from the proximal end thereof. A plurality of resilient prongs connected to a distal end of the shifter element, an actuator handle connected to the proximal end of the tubular member, and a shifting actuator slidably mounted to the handle and fixed to a proximal end of the shifter element.

### BACKGROUND OF THE INVENTION

This invention relates to a medical device useful in the treatment of hemorrhoids. More particularly, this invention relates to a medical device useful in the treatment of hemorrhoids by ligation. This invention also relates to an associated method.

It is a well-established medical procedure to place an elastic band about a hemorrhoid to cut off the blood supply to the hemorrhoid thereby resulting in necrosis of the hemorrhoidal tissues. The elastic band is initially disposed around a metal cylinder at the distal end of a ligator. The physician draws the hemorrhoid the cylinder by inserting the jaws of a forceps through the cylinder and manipulating the forceps to grasp the hemorrhoid and pull it into the cylinder. Operating an actuator at the handle end of the ligator, the physician shifts a second cylinder in a distal direction over the first cylinder, thereby pushing the elastic band off of the first cylinder and onto the hemorrhoid.

This hemorrhoid ligation operation can be difficult and requires skill and experience to complete quickly. One of the complications of the instrumentation is that the forceps must be precisely positioned relative to the ligator so that the joint between the forceps jaws is disposed inside the first cylinder when the jaws are opened, to maximize the separation of the jaws prior to placement about the hemorrhoid. The ligator and the opened forceps must then be moved in tandem towards the target hemorrhoid. In addition to these instruments, the physician must manipulate and hold an anoscope, which is inserted into the rectum at the onset of the operation.

### OBJECTS OF THE INVENTION

It is an object of the present invention to provide an improved instrument for grasping and drawing a hemorrhoid in a hemorrhoid ligation procedure.

It is another object of the present invention to provide such an instrument that is easy to use with conventional hemorrhoid ligators.

A further object of the present invention is to provide an improved hemorrhoid treatment method.

These and other objects of the present invention will be apparent from the drawings and descriptions herein. Although every object of the invention is believed to be attained in at least one embodiment of the invention, there is not necessary any single embodiment that achieves all of the objects of the invention.

### SUMMARY OF THE INVENTION

A medical grasper for treatment of hemorrhoids comprises, in accordance with the present invention, an elongate rigid tubular member having a proximal end and a distal end, an elongate shifter element extending longitudinally into the tubular member from the proximal end thereof, a plurality of resilient prongs connected to a distal end of the shifter element, an actuator handle connected to the proximal end of the tubular member, and a shifting actuator slidably mounted to the handle and fixed to a proximal end of the shifter element.

Pursuant to another feature of the present invention, the prongs each have a hook at a distal or free end, opposite the shifter element.

The prongs preferably each have an inherent spring bias or memory tending to form the prongs into arcuate shapes, each convex on an inner side facing one or more other prongs and concave on a side facing away from the one or more other prongs. In that case, the hooks may each include an inwardly oriented C- or U-shape.

In accordance with a further feature of the present invention, the grasper further comprise a spring member disposed between the handle and the shifting actuator for biasing the actuator to a distal position and concomitantly disposing the prongs in an exposed position, distally of the distal end of the tubular member.

Pursuant to additional features of the present invention, the handle member includes an elongate shaft provided with an elongate longitudinal slot, the shifting actuator being slidably mounted to the shaft. The shifter element extends longitudinally into the slot, while the shifting actuator is connected to the shifter element by a pin extending transversely into the slot. In this embodiment, the spring member may take the form of a compression spring disposed in the slot.

The handle may further include a thumb ring at a proximal end of the shaft, opposite the tubular member, while the shifting actuator takes the form of a spool. The thumb ring may be mounted to the shaft for rotation about a longitudinal axis of the shaft.

The tubular member of a medical grasper instrument in accordance with the present invention is preferably provided with a bend located between two inches and six inches from the distal end of the tubular member, and preferably between 3 inches and 5 inches from the distal end of the tubular member. A distance of about 4 inches is believed to be optimal.

A method for the treatment of hemorrhoids comprises, pursuant to the present invention, providing a grasper having an operative tip and a handle member with relatively slidable parts. The relatively slidable parts include a first part having an elongate shaft and a second part shiftable longitudinally along the shaft. The method also comprises providing a ligation instrument having a tubular shaft and a cylinder fixed to a distal tip of the shaft. The method further comprises manipulating the grasper to insert the operative tip thereof into the cylinder of the ligation instrument, manipulating the ligation instrument to place the cylinder near a hemorrhoid of a patient, further manipulating the grasper to place the operative tip adjacent the hemorrhoid, thereafter sliding the first part longitudinally along the elongate shaft of the second part to operate the operative tip to close the operative tip about the hemorrhoid, thereafter further manipulating the grasper to draw the hemorrhoid into the cylinder, and subsequently sliding an elastic band off of the cylinder and onto the hemorrhoid.

Where the handle member includes a thumb ring, the sliding of the first part along the elongate shaft includes operating the grasper with one hand, inserting a thumb through the thumb ring and manipulating the first part with at least one finger to move the first part towards the thumb ring.

Where the operative tip of the grasper includes a plurality of resilient prongs, the grasper includes a tube connected to the handle member, and the prongs extend in a separated configuration prior to contact with the hemorrhoid, the sliding of the first part along the elongate shaft draws the prongs into a distal end of the tube to close the prongs about the hemorrhoid.

### BRIEF DESCRIPTION OF THE DRAWINGS

FIG. 1 is a side elevational view of a hemorrhoid grasper in accordance with the present invention.

FIG. 2 is a top plan view of the hemorrhoid grasper of FIG. 1.

FIG. 3 is a perspective view of the hemorrhoid grasper of FIGS. 1 and 2.

FIG. 4 is a side elevational view of the grasper of FIGS. 1-3 and a hemorrhoid ligator, showing a step in the use of the hemorrhoid grasper in a hemorrhoid ligation procedure.

FIG. 5 is a view similar to FIG. 4, showing another step in the use of the hemorrhoid grasper of FIGS. 1-3 in a hemorrhoid ligation procedure.

### DETAILED DESCRIPTION

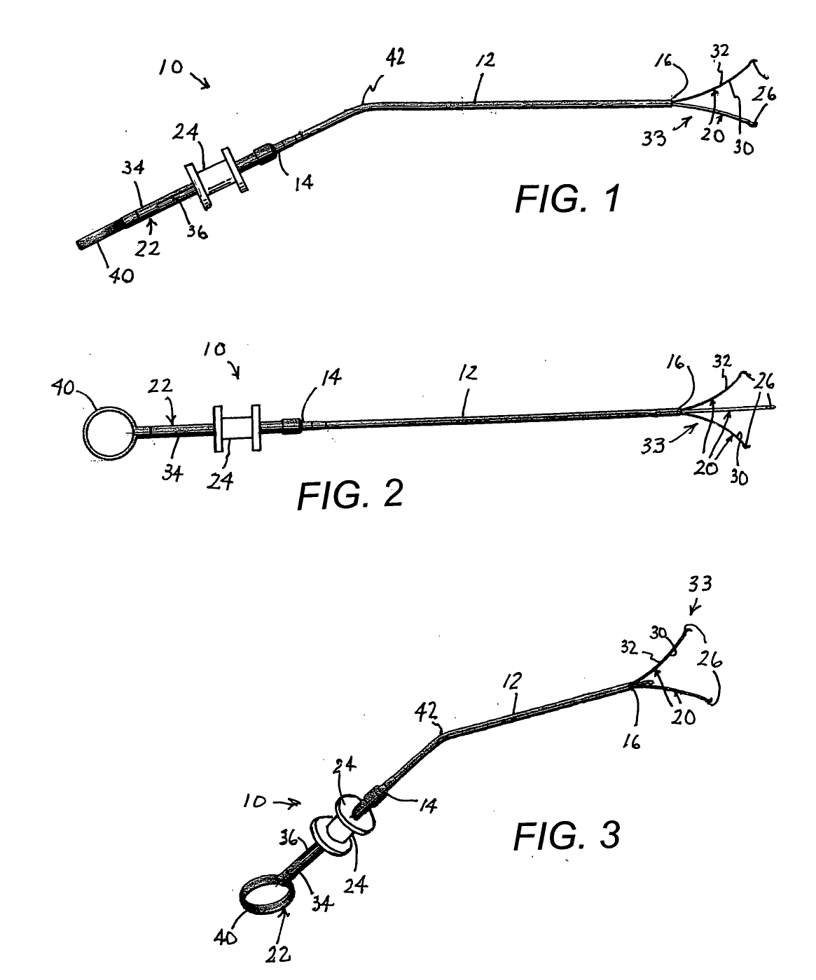

As depicted in FIGS. 1-3, a medical grasper 10 for the treatment of hemorrhoids comprises an elongate rigid tubular member 12 having a proximal end 14 and a distal end 16. An elongate shifter element 18 (FIG. 5) in the form of a stiff wire extends longitudinally into tubular member 12 from proximal end 14 thereof. A plurality of resilient prongs 20 are connected to a distal end of shifter element 18, while an actuator handle 22 is connected to proximal end 14 of tubular member 12. A shifting actuator 24 is slidably mounted to handle 22 and fixed to a proximal end of shifter element 18.

Prongs 20 each have a hook 26 at a distal or free end, opposite shifter element 18. Hooks 26 each include an inwardly oriented C- or U-shape. Each prong 20 has an inherent spring bias or memory tending to form the prong into arcuate shape, convex on an inner side 30 facing the other prong or prongs and concave on a side 32 facing away from the other prong(s). Prongs 20 collectively comprise an operative tip 33 of grasper 10

Handle member 22 includes an elongate shaft 34 provided with an elongate longitudinal slot 36. Shifting actuator 24 is slidably mounted to shaft 34. Shifter element or wire 18 extends longitudinally into slot 36, while shifting actuator 24 is connected to shifter element 18 by a coupling pin 38 extending transversely into the slot. Shifter element or wire 18 may be enlarged at a proximal end to facilitate connected to coupling pin 38.

Grasper 10 further comprise a compression spring member 34 disposed in slot 36 between between handle 22 and shifting actuator 24 for biasing the actuator to a distal position and concomitantly disposing prongs 20 in an exposed position (FIGS. 1-3), distally of the distal end 16 of tubular member 12.

Handle 22 further include a thumb ring 40 at a proximal end of shaft 34, opposite tubular member 12, and shifting actuator 24 takes the form of a spool. Thumb ring 40 is mounted to the proximal end of shaft 34 for rotation about a longitudinal axis of the shaft.

As shown in FIGS. 1 and 3, tubular member 12 is provided with a bend 42 located between two inches and six inches from the distal end 16 of the tubular member, and preferably between 3 inches and 5 inches from the distal end of the tubular member. A distance of about 4 inches is believed to be optimal.

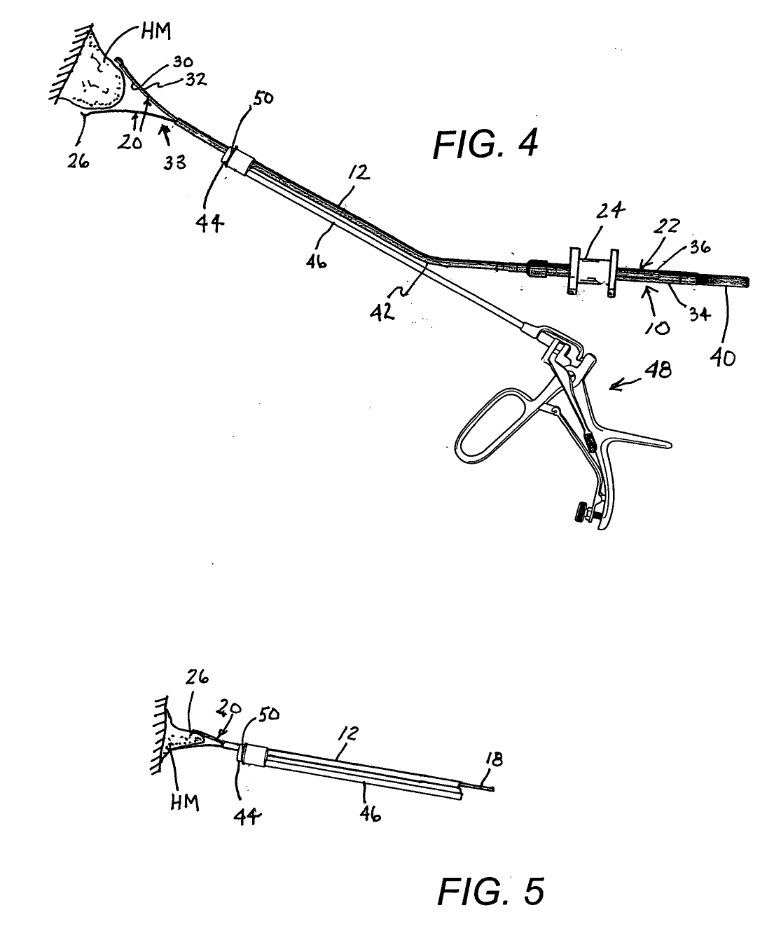

In a hemorrhoidal treatment operation, one manipulates grasper 10 to insert operative tip 33 through a band-carrying cylinder 44 of a ligator 48, as shown in FIG. 4. Ligator 48 includes a rigid hollow shaft 46, cylinder 44 being fixed to a distal tip of the shaft. One manipulates the ligator 48 to place cylinder 44 near a hemorrhoid HM of a patient. Grasper 10 is further manipulated to place operative tip 33 adjacent the hemorrhoid HM (FIG. 4). Operative tip 33 and distal end 16 of tubular member 12 may be inserted through cylinder 44 prior to or after the juxtaposition of the cylinder with the hemorrhoid HM. After the placement of the operative tip 33 adjacent the hemorrhoid, so that prongs 20 are on different sides of the hemorrhoidal tissues, the surgeon slides shifting actuator or spool 24 longitudinally along shaft 34 of the handle 22 to retract prongs 20 into distal end 16 of tubular member 12, thereby closing the operative tip 33 about the hemorrhoid HM (FIG. 5). Thereafter the surgeon further manipulates grasper 10 to draw the hemorrhoid HM into the cylinder 44 and subsequently operates ligator 48 to slide an elastic band 50 off of the cylinder and onto the hemorrhoid.

The sliding of the shifting actuator or spool 24 along shaft 34 includes operating the grasper 10 with one hand, inserting a thumb (not shown) through thumb ring 40 and manipulating shifting actuator or spool 24 with at least one finger to move the actuator or spool towards thumb ring 40.

Upon the application of the elastic band 50 to the hemorrhoid, shifting actuator or spool 24 is translated in the distal direction, away from thumb ring 40, to thereby open operative tip 33 and separate the prongs 20 from each other. Hooks or teeth 26 are disengaged from the hemorrhoid HM, whereupon both ligator 48 and grasper 10 are removed from the rectal area.

The above described hemorrhoid treatment procedure typically involves the use of ancillary instrument including an anoscope, a tubular member that props upon the anus to facilitate visual and operative access to the surgical site.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A medical grasper for treatment of hemorrhoids, comprising: an elongate rigid tubular member having a proximal end and a distal end; an elongate shifter element extending longitudinally into said tubular member from said proximal end thereof; a plurality of resilient prongs connected to a distal end of said shifter element; an actuator handle connected to said proximal end of said tubular member; and a shifting actuator slidably mounted to said handle and fixed to a proximal end of said shifter element.

2. The grasper defined in claim 1 wherein said prongs each have a hook at a distal or free end, opposite said shifter element.

3. The grasper defined in claim 2 wherein said prongs have an inherent spring bias or memory tending to form said prongs into arcuate shapes.

4. The grasper defined in claim 3 wherein said prongs are each convex on an inner side facing one or more other prongs and concave on a side facing away from the one or more other prongs, the hooks each including an inwardly oriented C- or U-shape.

5. The grasper defined in claim 1, further comprising a spring member disposed between said handle and said shifting actuator for biasing said actuator to a distal position, whereby said prongs are disposed in an exposed position, distally of the distal end of said tubular member.

6. The grasper defined in claim 5 wherein said handle member includes an elongate shaft provided with an elongate longitudinal slot, said shifting actuator being slidably mounted to said shaft, said shifter element extending longitudinally into said slot, said shifting actuator being connected to said shifter element by a pin extending transversely into said slot, said spring member being a compression spring disposed in said slot.

7. The grasper defined in claim 6 wherein said handle further includes a thumb ring at a proximal end of said shaft, opposite said tubular member.

8. The grasper defined in claim 7 wherein said thumb ring is mounted to said shaft for rotation about a longitudinal axis of said shaft.

9. The grasper defined in claim 7 wherein said shifting actuator takes the form of a spool.

10. The grasper defined in claim 1 wherein said tubular member has a bend located between said proximal end and the distal end of said tubular member.

11. The grasper defined in claim 10 wherein said bend is located between three inches and 5 inches from the distal end of said tubular member.

12. A method for the treatment of hemorrhoids, comprising: providing a grasper having an operative tip and a handle member with relatively slidable parts including a first part having an elongate shaft and a second part shiftable longitudinally along said shaft; providing a ligation instrument having a tubular shaft and a cylinder fixed to a distal tip of said shaft; manipulating said grasper to insert said operative tip through said cylinder; manipulating said ligation instrument to place said cylinder near a hemorrhoid of a patient; further manipulating said grasper to place said operative tip adjacent said hemorrhoid; sliding said first part longitudinally along said elongate shaft of said second part to close said operative tip about said hemorrhoid; after the closure of said operative tip about said hemorrhoid, further manipulating said grasper to draw said hemorrhoid into said cylinder; and subsequently sliding an elastic band off of said cylinder and onto said hemorrhoid.

13. The method defined in claim 12 wherein said handle member includes a thumb ring, the sliding of said first part along said elongate shaft includes inserting a thumb of one hand through said thumb ring and manipulating said first part with at least one finger of said one hand to move said first part towards said thumb ring.

14. The method defined in claim 13 wherein said operative tip includes a plurality of resilient prongs, said grasper including a tube connected to said handle member, said prongs extending in a separated configuration prior to contact with said hemorrhoid, the sliding of said first part along said elongate shaft drawing said prongs into a distal end of said tube to close said prongs about said hemorrhoid.
